(19) United States
(12) Patent Application Publication
(10) Pub. No.: US 2002/0052631 A1
(43) Pub. Date: May 2, 2002

(54) Method and apparatus using a time measurement to an electrical parameter threshold to determine a defibrillation pulse duration

(76) Inventors: Joseph L. Sullivan, Kirkland, WA (US); Daniel W. Piraino, Seattle, WA (US); Gary DeBardi, Kirkland, WA (US); Richard C. Nova, Kirkland, WA (US); Fred W. Chapman, Renton, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 09/885,543

(22) Filed: Jun. 19, 2001

Related U.S. Application Data
(60) Provisional application No. 60/213,082, filed on Jun. 19, 2000.

(51) Int. Cl. A61N 1/39

(52) U.S. Cl. 607/7

(57) ABSTRACT

A defibrillator method and apparatus use a measurement of the time that it takes for an electrical parameter related to the defibrillation pulse discharge to reach an intermediate parameter threshold to determine the duration of the pulse. In one embodiment, the defibrillator measures the elapsed time that it takes for an electrical parameter to reach a predetermined intermediate parameter threshold. The defibrillator determines a period of time for extending the duration of the pulse based on the measured elapsed time. In other embodiments, the intermediate parameter threshold may vary during the pulse discharge and/or multiple intermediate parameter thresholds may be used. The measurement of elapsed time may also be used to determine other conditions for truncating the defibrillation pulse, such as voltage, current, energy, charge, or time. The defibrillator thus provides a defibrillation pulse whose duration is dynamically determined from a time measurement made during the delivery of the pulse.

### CROSS-REFERENCE TO RELATED APPLICATION

This application claims the benefit of the filing date of U.S. Provisional Application No. 60/213,082, filed Jun. 19, 2000, under the provisions of 35 U.S.C. § 119(e).

### FIELD OF THE INVENTION

The invention relates generally to cardiac defibrillation and, more particularly, to determining the duration of defibrillation pulses delivered to patients having different physiological characteristics.

### BACKGROUND OF THE INVENTION

Cardiac fibrillation is a medical condition in which the electrical activity of a patient's heart becomes unsynchronized, resulting in the loss of the heart's ability to contract and pump blood into the circulation system. Both atrial and ventricular fibrillation may be treated by applying an electric pulse to the heart that is strong enough to stop the unsynchronized electrical activity and give the heart a chance to reinitiate a synchronized rhythm. External defibrillation is a method of applying an electric pulse to a patient's fibrillating heart from the surface of the patient's body. Other cardiac arrhythmias, such as ventricular tachycardia, may also be converted to a normal heart rhythm by applying an electric pulse of appropriate amplitude and duration to the heart.

A defibrillation pulse applied to a patient encounters a resistance to the flow of electrical current through the patient. The resistance of a patient's body to the flow of electrical current is known as transthoracic impedance (typically measured in ohms). External defibrillators may be used with different patients having different physiological characteristics, and thus are likely to encounter patients having a wide range of transthoracic impedance. Conventional defibrillators are often specified for, and tested with, a 50 ohm load (which, in many cases, is selected to represent a “standard” patient). However, the actual transthoracic impedance of human patients can vary greatly in a range of 20 to 300 ohms, though most patients typically fall in a range of 25 to 180 ohms.

Defibrillation pulses that are effective in treating low impedance patients may not necessarily deliver effective and efficient treatment to high impedance patients, and vice versa. Accordingly, one challenge faced by defibrillator manufacturers is to design defibrillators that work well over a wide range of patients.

Some prior art defibrillators have attempted to address the variability of patient impedance by adjusting the duration of the waveforms according to the patient's impedance. For example, U.S. Pat. No. 5,230,336 to Fain et al., discloses a method of selecting a suggested pulse width for a second defibrillation pulse based on system impedance measured during delivery of a first defibrillation pulse. Other prior art techniques, e.g., U.S. Pat. No. 6,047,212 to Gliner et al., involve specific algorithms for dynamic adjustment of the truncation point of a defibrillation pulse in response to different patient impedances. U.S. Pat. No. 5,540,723 to Ideker et al. discloses a method and apparatus for treating cardiac arrhythmias wherein the duration of a defibrillation pulse is adjusted in accordance with a patient model and pulse signal time constant to coincide with the peak amplitude of the patient model response. U.S. Pat. No. 5,352,239 to Pless discloses apparatus that immediately truncates a defibrillation pulse when the amplitude of the pulse decays to a programmed voltage.

Despite the teachings of the prior art, there remains a need for more advantageous ways to determine the pulse duration during discharge to provide the patient more effective and efficient therapy. The present invention is directed to addressing this need and other shortcomings in the prior art.

### SUMMARY OF THE INVENTION

The present invention is directed to a defibrillator method and apparatus that use a time measurement for an electrical parameter to reach an intermediate parameter threshold to determine the duration of the pulse. The defibrillator includes an energy storage device, electrodes in electrical communication with the energy storage device, and a processing unit configured to cause the energy storage device to discharge energy in a defibrillation pulse through the electrodes. The energy discharge continues until a condition for truncating the discharge is met. The condition for truncating the energy discharge is determined based on the elapsed time for the electrical parameter to reach the intermediate parameter threshold.

In one embodiment of the invention, the processing unit measures the time it takes for an electrical parameter related to the pulse discharge, such as voltage, current, energy, or charge, to reach a predetermined intermediate parameter threshold. Based on the measured elapsed time, the processing unit determines a period of time for extending the duration of the pulse discharge. The electrical parameter related to the discharge may be an instantaneous measurement of the parameter, or may be an averaged or summed value, as desired. In this manner, the present invention provides a time-terminated defibrillation pulse whose duration is based on a time measurement made during the delivery of the pulse.

The intermediate parameter threshold may be predetermined based on factors such as a charge level of the energy storage device or a patient-related parameter such as impedance. The patient's impedance may be calculated during the pulse delivery based on an initial value of the electrical parameter, the intermediate parameter threshold, and the measured elapsed time for the electrical parameter to reach the intermediate parameter threshold. The patient's impedance may also be determined prior to the defibrillation pulse delivery based on information obtained in an earlier defibrillation pulse delivered to the patient, if any, and/or information obtained by transmitting a low-amplitude signal through the patient. If the calculated patient impedance (or other patient-related parameter) is outside an expected range, the processing unit may further be configured to test an aspect of the defibrillator and/or advise an operator of the defibrillator of a possible error in the energy delivery.

The processing unit may determine the period of time for extending the duration of the pulse discharge from a look-up table containing duration values stored in memory. The processing unit may use the measured elapsed time as an index to the look-up table to determine the remaining pulse duration.

In another embodiment of the invention, the intermediate parameter threshold varies during the pulse discharge. The defibrillator measures an elapsed time for an electrical parameter related to the discharge to reach the variable intermediate parameter threshold, and extends the duration of the energy discharge for a period of time that is determined based on the measured elapsed time. The intermediate parameter threshold may vary based on the elapsing discharge time. The intermediate parameter threshold may also vary according to an anticipated time for the electrical parameter to reach the intermediate parameter threshold and a desired energy discharge duration so that the intermediate parameter threshold is reached at least one millisecond before the end of the desired energy discharge duration.

The initial value of the variable intermediate parameter threshold may be set based on an impedance of the patient. As noted earlier, the patient's impedance may be determined prior to delivery of the defibrillation pulse from information obtained during delivery of a prior defibrillation pulse to the patient, and/or from a low-amplitude signal transmitted through the electrodes attached to the patient.

Another embodiment of the invention measures a first elapsed time for a first electrical parameter related to the defibrillation pulse discharge to reach a first intermediate parameter threshold. If the first elapsed time is within an expected range of time, the duration of the energy discharge is extended for a period of time that is determined based on the first elapsed time. Otherwise, the energy discharge is continued and the elapsed time for a second electrical parameter related to the discharge to reach a second intermediate parameter threshold is measured. If the second elapsed time is within an expected range of time, the duration of the energy discharge is extended for a period of time that is determined based on the second elapsed time. Otherwise, the energy discharge is continued and the process of measuring an elapsed time for an electrical parameter to reach an intermediate parameter threshold is repeated until either (1) the measured elapsed time is within an expected range of time, after which the duration of the energy discharge is extended for a period of time that is determined based on the measured elapsed time, or (2) the process of measuring the elapsed time to an intermediate threshold has been repeated for a determined number of iterations, after which the duration of the energy discharge is extended for a default period of time. The first and second electrical parameters may be the same parameter or different parameters. The processing unit of the defibrillator may be further configured to automatically proceed to measuring the second elapsed time if the first elapsed time exceeds the first expected range of time, without waiting for the first electrical parameter to reach the first intermediate parameter threshold. The first or second intermediate parameter thresholds may also vary during the energy discharge based on the elapsing discharge time.

In yet another embodiment of the invention, a processing unit measures an elapsed time for an initial electrical parameter related to the discharge to reach an initial intermediate parameter threshold. While continuing the energy discharge, the processing unit repeats a process of determining a subsequent electrical parameter and subsequent intermediate parameter threshold that is determined based on the elapsed time for the previous electrical parameter to reach the previous intermediate parameter threshold. This process is repeated until a final electrical parameter reaches a final intermediate parameter threshold, at which time the duration of the energy discharge is extended for a period of time that is determined based on the elapsed time for the final electrical parameter to reach the final intermediate parameter threshold. In this embodiment of the invention, the initial or subsequent intermediate parameter thresholds may vary based on the elapsing time. The value of the initial or subsequent intermediate parameter thresholds may be set based on an impedance of the patient or based on a charge level of the defibrillator's energy storage device. The processing unit may be configured to determine the period of time for extending the duration of the energy discharge from a look-up table containing duration values stored in the defibrillator's memory. For example, the discharge duration may be extended from the final intermediate parameter threshold by using the elapsed time for the final electrical parameter to reach the final intermediate parameter threshold as an index to the look-up table.

In further embodiments of the invention, the defibrillator may use the measurement of elapsed time to determine other conditions for truncating the energy discharge. For example, based on the measured elapsed time, the defibrillator may set a voltage parameter that, when reached by a voltage related to the energy discharge, causes truncation of the energy discharge. Other conditions for truncating the energy discharge may be based on a current parameter, energy parameter, charge parameter, or time parameter. When the condition for truncating the energy discharge is a time parameter, for example, the condition may be an end time for the energy discharge.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a pictorial illustration of an external defibrillator constructed in accordance with the present invention as attached to a patient;

FIG. 2 is a strength-duration graph illustrating the effect of amplitude and duration of a defibrillation pulse on successful defibrillation;

FIG. 3 is a graph depicting a biphasic truncated exponential defibrillation pulse delivered in accordance with the present invention;

FIG. 4A is the first part of a flow diagram illustrating a method of delivering a biphasic defibrillation pulse from the defibrillator shown in FIG. 1 in accordance with the present invention;

FIG. 4B is the second part of a flow diagram illustrating a method of delivering a biphasic defibrillation pulse in accordance with the present invention;

FIG. 5 is a pictorial diagram of a look-up table stored in a memory of the defibrillator shown in FIG. 1 which correlates measured elapsed time to an intermediate parameter threshold for determining the remaining pulse duration;

FIG. 6 is the first part of a flow diagram illustrating an alternative method of delivering a biphasic defibrillation pulse, the second part being provided by FIG. 4B;

FIG. 7 is a block diagram illustrating major components of a defibrillator as shown in FIG. 1 configured in accordance with the present invention; and

FIG. 8 is the first part of a flow diagram illustrating an alternative method of delivering a biphasic defibrillation pulse, the second part being provided by FIG. 4B.

### DETAILED DESCRIPTION OF THE PREFERRED EMBODIMENT

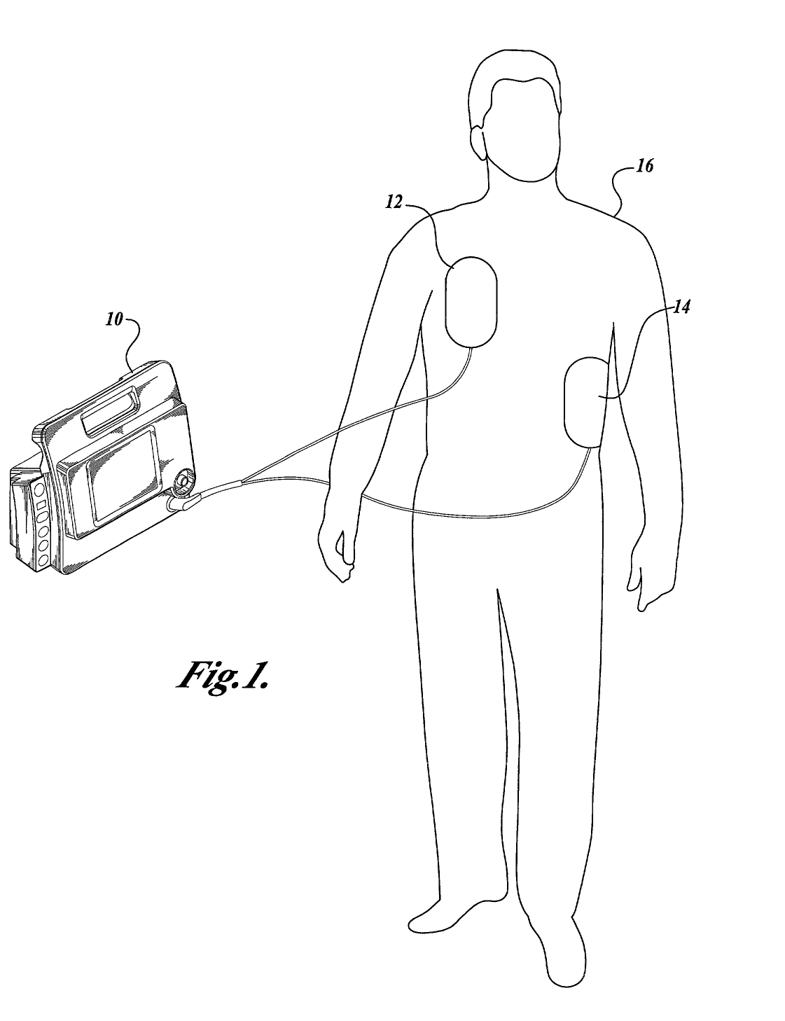

A defibrillator is an energy delivery system that discharges energy to a patient in the form of a defibrillation pulse. FIG. 1 depicts an external defibrillator 10 configured to deliver a defibrillation pulse in accordance with the present invention. The defibrillator 10 is shown attached to a patient 16 via a plurality of disposable defibrillation electrodes 12 and 14. More specifically, and as will be discussed in greater detail below, the defibrillator 10 delivers to the patient 16 a defibrillation pulse having a duration that depends on a measurement of the time it takes for an electrical parameter related to the defibrillation pulse to reach an intermediate threshold value.

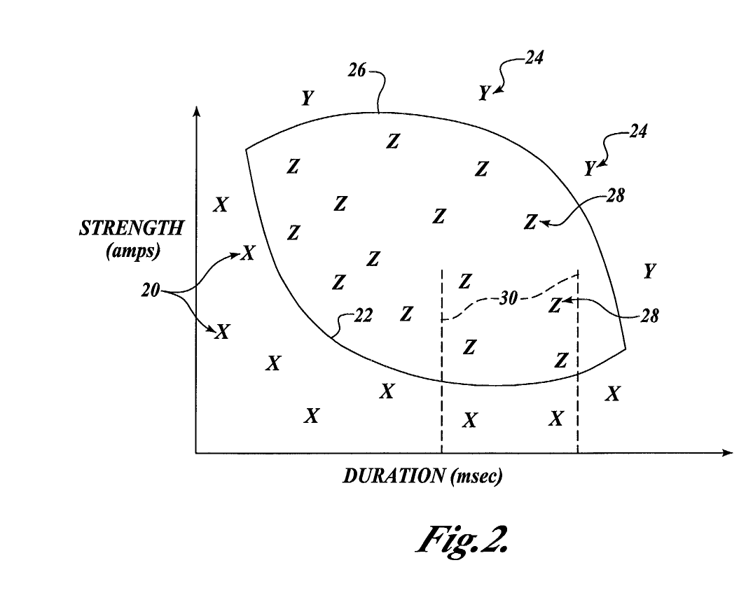

Prior to discussing various exemplary embodiments of the present invention, it is worthwhile to note that the duration of a defibrillation pulse directly affects the success of the defibrillation pulse in defibrillating a patient's heart. A strength-duration graph, as shown in FIG. 2, illustrates the effect on successful defibrillation of varying the amplitude and duration of a defibrillation pulse. The strength (amplitude) of a defibrillation pulse, shown in FIG. 2 as amperes of current, is plotted on the vertical axis while the duration of the pulse, shown in terms of milliseconds, is plotted on the horizontal axis.

For a given pulse strength and duration, there is a probability that the pulse will succeed in terminating fibrillation of the patient's heart. Defibrillation pulses having too little strength or too little duration, as noted by the X's 20, are likely to fail in terminating fibrillation. A minimum strength-duration threshold, shown by curve 22, identifies the crossover from defibrillation pulses that are less likely to more likely result in successful defibrillation. Defibrillation pulses having too great strength and/or too long duration, as indicated by the Y's 24, are likely to physiologically damage a patient and/or cause the patient's heart to refibrillate. A maximum strength-duration threshold, shown by curve 26, identifies the crossover from successful defibrillation pulses to pulses that are more likely to cause damage or refibrillate the heart. Accordingly, there is a range of pulse strength and duration, as shown by the Z's 28, in which a defibrillation pulse is more likely to succeed in defibrillating a heart while avoiding long-term damage or refibrillation. A pulse duration that minimizes the strength needed for successful defibrillation lies in the range between the dashed lines 30.

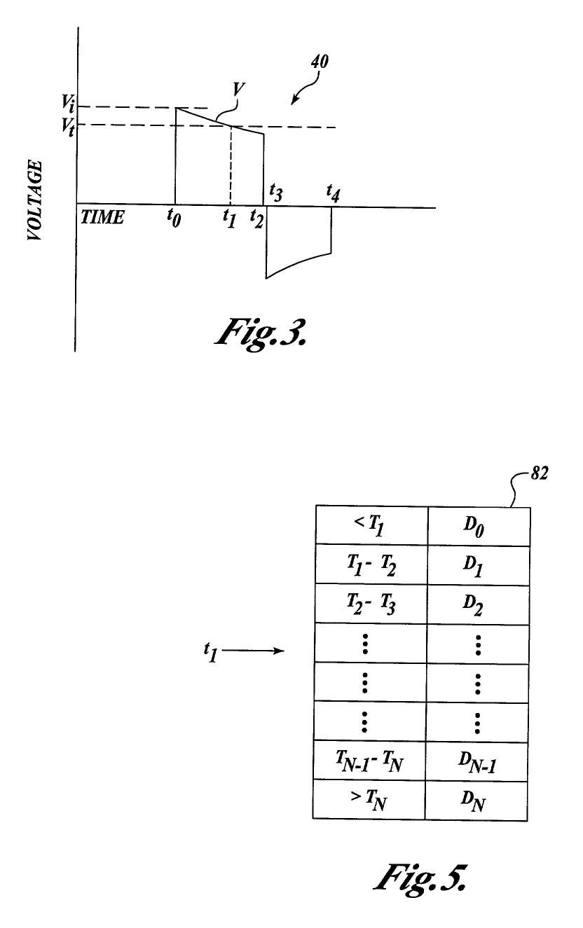

The present invention, in its various embodiments, provides methods and apparatus for delivering a defibrillation pulse in which the duration of the pulse is determined based on an elapsed time in which it takes an electrical parameter related to the energy discharge to reach an intermediate parameter threshold. FIG. 3 illustrates a biphasic truncated exponential defibrillation pulse 40 delivered to a patient in accordance with the present invention. While the defibrillation pulse 40 is a biphasic pulse, the present invention is also applicable to single phase or other multiphasic defibrillation pulses. Where a multiphasic pulse is concerned, references herein to extending the duration of the defibrillation pulse should be understood as applicable to extending the duration of one or more of the pulse phases. In terms of the biphasic pulse 40 shown in FIG. 3, the duration of the first phase is extended for a period of time (from time t₁ to t₂) that is determined based on the elapsed time it takes (from time t₀ to t₁) for the pulse voltage V to reach the intermediate threshold voltage Vₜ, as will be more readily understood from the description below.

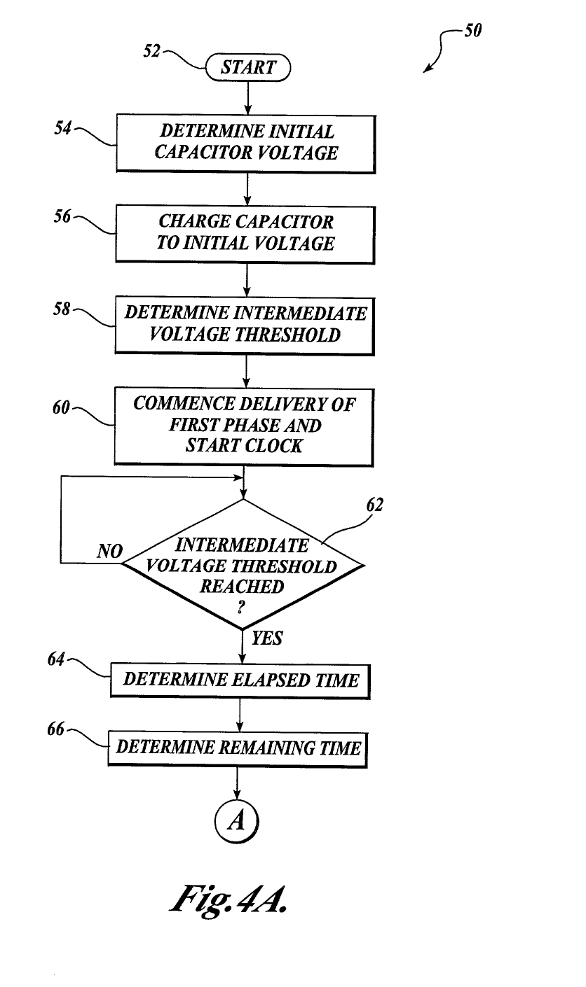

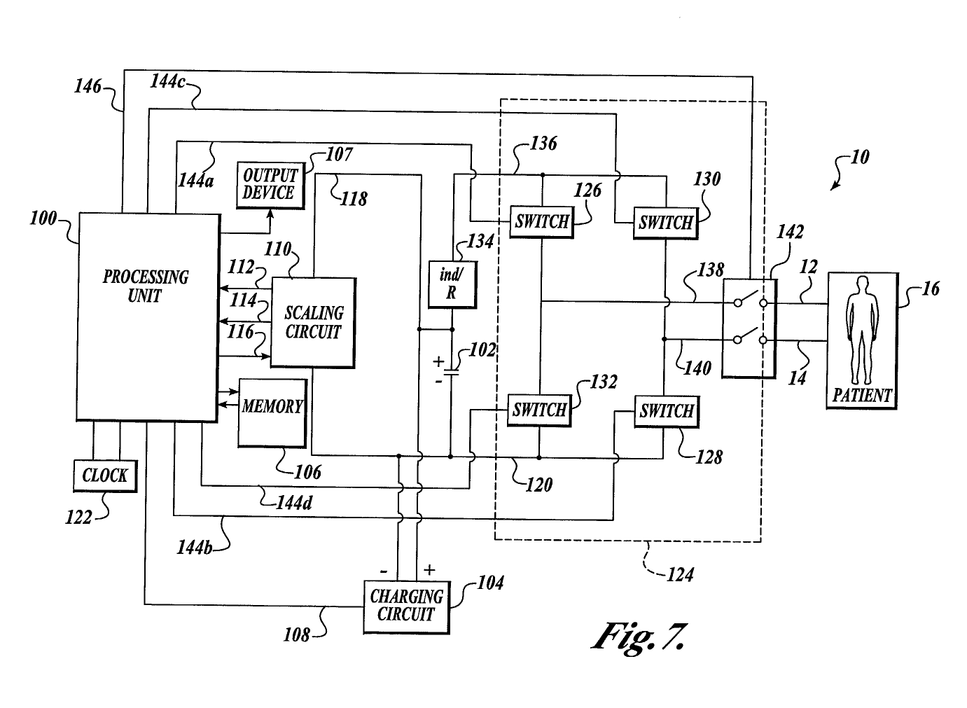

FIGS. 4A and 4B provide a flow diagram illustrating a method 50 of delivering a biphasic defibrillation pulse, as shown in FIG. 3, in accordance with one embodiment of the present invention. Turning specifically to FIG. 4A, the method 50 begins at a start block 52. At block 54, the defibrillator 10 determines the initial charge level for charging an energy storage device that is used to deliver the defibrillation pulse to the patient. In this particular embodiment, the defibrillator 10 determines the initial voltage to which an internal defibrillation capacitor (e.g., capacitor 102 in FIG. 7, discussed below) should be charged to deliver the defibrillation pulse.

The initial charge level (in this case, initial capacitor voltage) may be set in accordance with factors such as the energy setting of the defibrillator (often selected by an operator of the defibrillator) and/or a measurement of the patient's transthoracic impedance. One suitable method for determining the initial capacitor voltage is discussed in U.S. Pat. No. 5,999,852, titled “Defibrillator Method and Apparatus,” assigned to the assignee of the present invention and specifically incorporated by reference herein. As described in U.S. Pat. No. 5,999,852, the initial capacitor voltage is determined in accordance with the energy setting of the defibrillator and a measurement of the patient's impedance obtained prior to delivery of the defibrillation pulse using a low-amplitude AC signal transmitted through the patient 16 via the electrodes 12 and 14.

In an alternative embodiment of the invention in which the defibrillator 10 uses external and/or internal hard paddles instead of the disposable defibrillation electrodes 12 and 14 shown in FIG. 1. The initial capacitor voltage is determined based on the energy setting alone. Other embodiments of the invention may set the initial charge level of the capacitor in terms of the joules of energy stored or other suitable units of measure. In any event, in this embodiment, the defibrillator 10 charges its defibrillation capacitor to the determined initial capacitor voltage at block 56. The energy source for charging the defibrillation capacitor may be internal or external to the defibrillator.

At block 58, the defibrillator 10 determines an intermediate electrical parameter threshold, such as an intermediate capacitor voltage threshold Vₜ (FIG. 3), that, when met during delivery of the defibrillation pulse, causes the defibrillator to observe the elapsed time that it took the electrical parameter to reach the intermediate threshold. The elapsed time is then used to determine a period of time for extending the duration of the pulse delivery, as discussed below in regard to block 66. The elapsed time can be measured from any point earlier in the pulse (e.g., the start of the pulse delivery).

In one embodiment of the invention, the intermediate parameter threshold is predetermined prior to delivering the defibrillation pulse to the patient. Alternatively, the intermediate threshold may be set after the pulse delivery has commenced based on observed characteristics of the defibrillation pulse. In either case, as discussed herein, the intermediate parameter threshold may be maintained throughout the delivery of the pulse, or the intermediate parameter threshold may be varied during the pulse delivery.

The intermediate parameter threshold may be predetermined based on a percentage of a charge level of the energy storage device. Where the electrical parameter is capacitor voltage, for example, the intermediate parameter threshold in this embodiment of the invention may be set as percentage of the initial voltage of the charged defibrillation capacitor. It is not required that the defibrillation capacitor be fully charged prior to determining the intermediate parameter threshold. In terms of the method 50 in FIG. 4A, the intermediate voltage threshold (block 58) may be determined at the same time that the initial capacitor voltage is determined (block 54).

An advantage of setting the intermediate voltage threshold, for example, as a percentage of the initial charge voltage is that the procedure for determining the time period for extending the pulse duration after the intermediate voltage threshold is met (which may include the use of a time-indexed look-up table described below) can be made independent of the energy setting of the defibrillator. In alternative embodiments of the invention, the intermediate parameter threshold may be determined based on other factors, such as the energy settings of the device, the patient's impedance, and/or the type of cardiac rhythm that is present in the patient. For instance, a combination of the patient's impedance and the selected energy setting for the patient could be used as factors in an equation to determine the intermediate parameter threshold for the patient.

When using an intermediate voltage threshold that is set as a percentage of the initial capacitor voltage, the percentage is preferably high enough so that the threshold will be reached within a desired time during delivery of the pulse, regardless of patient impedance. If patient impedance is known, the percentage used may vary according to the patient impedance. Preferably, the capacitor voltage is not allowed to drop as much during pulse delivery to a high impedance patient as it may when delivered to a low impedance patient. For a 300-ohm patient, for example, the intermediate voltage threshold should be set at a relatively high level. By comparison, for a 20-ohm patient, the capacitor voltage during discharge will decay much quicker, and the threshold may be set at a lower level. In either circumstance, the time that it takes the voltage of the defibrillation capacitor (as the selected electrical parameter in this example) to reach the intermediate voltage threshold is used to determine the remaining pulse duration.

Returning to FIG. 4A, the defibrillator 10 commences at block 60 to deliver the defibrillation pulse to the patient. In that respect, the defibrillator may visually or audibly prompt the operator of the defibrillator to press a button to initiate delivery of the pulse. In other embodiments of the invention, the defibrillator may immediately commence delivery of the defibrillation pulse without operator interaction.

During the delivery of the defibrillation pulse to the patient, the defibrillator 10 observes a clock to measure the duration of the defibrillation pulse. In terms of the biphasic defibrillation pulse 40 shown in FIG. 3, the defibrillation pulse commences at time t₀, at which time the initial charge voltage of the defibrillation capacitor, and hence the initial pulse amplitude, is equal to Vᵢ. Subsequent to time to, the amplitude V of the defibrillation pulse begins to decay exponentially as the pulse is delivered to the patient. As demonstrated by decision block 62 in FIG. 4A, the defibrillator continues to deliver the defibrillation pulse to the patient until the voltage of the pulse reaches intermediate voltage threshold Vₜ.

Once the pulse voltage V has decayed to the intermediate voltage threshold Vₜ, the defibrillator 10 determines the elapsed time for the pulse voltage to reach the intermediate voltage threshold, as indicated at block 64. In terms of the pulse waveform shown in FIG. 3, the elapsed time from the beginning of pulse equals t₁ minus t₀. If time to is set to equal zero, time t₁ itself is a measure of the elapsed time for the pulse amplitude V to decay to the intermediate voltage threshold Vₜ. Other embodiments of the invention may suitably measure an elapsed time from another time point other than the start of the pulse delivery.

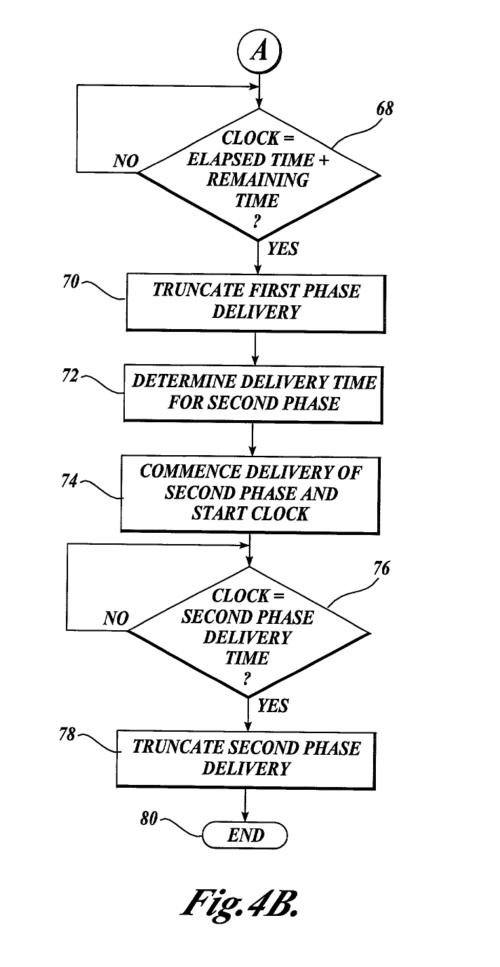

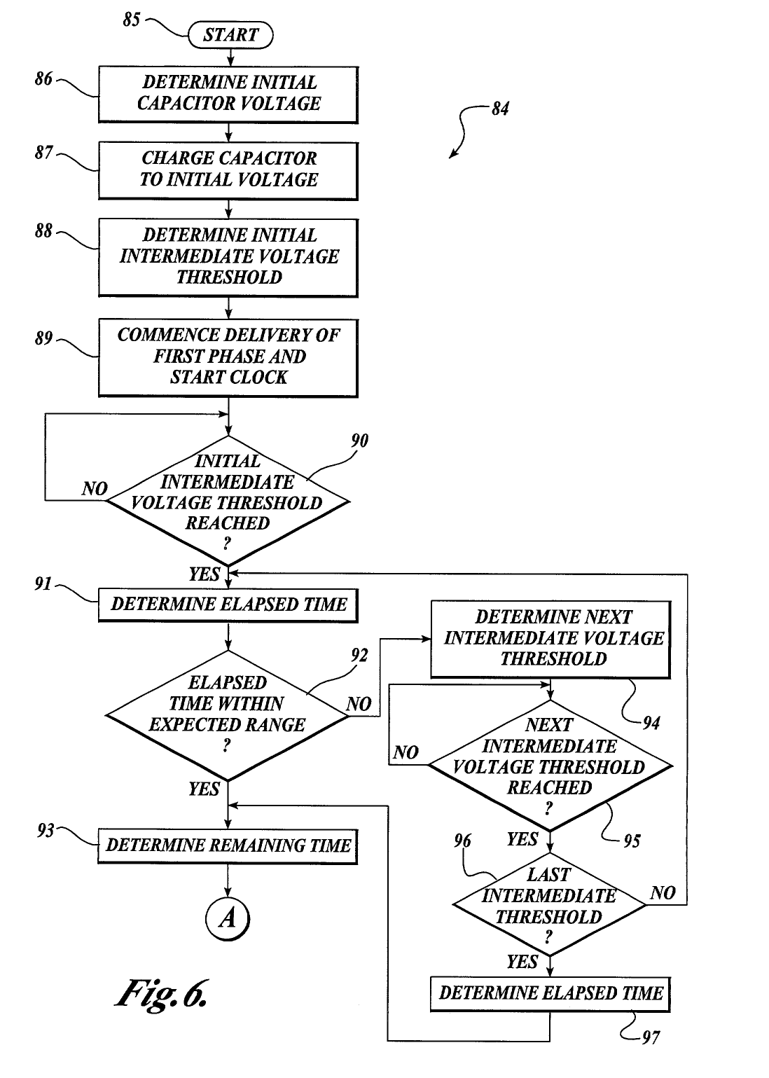

To determine the remaining delivery time for the first phase of the defibrillation pulse in this embodiment of the invention, as indicated at block 66, the elapsed time value t₁ is applied to a predetermined look-up table stored in the defibrillator's memory (e.g., memory 106 shown in FIG. 6). A pictorial illustration of a suitable look-up table 82 is provided in FIG. 5. The look-up table 82 correlates duration values D₀, D₁, D_{2,} . . . D_{N-1}, D_{N} with varying ranges of elapsed time values T. For instance, if the elapsed time t₁ is less than time value T₁, the look-up table 82 returns the remaining duration time D₀. If the elapsed time value t₁ falls within the range of time values T₁-T₂, the look-up table 82 returns the remaining duration time D₁, etc. The defibrillator uses the elapsed time t₁ as an index to the table 82 to identify the time period for extending the duration of the defibrillation pulse. While an interpolation algorithm can be used to determine a more specific duration value D for each possible elapsed time t₁, it is acceptable to use ranges of time values T, as shown in FIG. 5. The manner in which the duration values D are predetermined and stored in the look-up table in memory 106 is described below.

In one embodiment of the invention, the remaining duration values D are derived in a preprocessing stage wherein a patient's physiological response to a defibrillation pulse is modeled by the response of a parallel resistor-capacitor circuit. The amplitude of a defibrillation pulse delivered to a patient decays over a period of time in accordance with a “time constant” equivalent to the product of the capacitance and the resistance of the defibrillator system (as attached to the patient). The capacitance of a defibrillator system is dictated mostly by the size and configuration of the defibrillation capacitor (or capacitors) in the defibrillator. The resistance in a defibrillator system is generally the sum of both the defibrillator's internal resistance (which is typically small) and the resistance of the patient.

In an article titled “Choosing the Optimal Monophasic and Biphasic Waveforms for Ventricular Defibrillation,” Journal of Cardiovascular Electrophysiology, September 1995, specifically incorporated by reference herein, the authors Walcott et al. describe a mathematical model that predicts optimal truncation points for monophasic and first phase of biphasic waveforms. The optimal truncation point varies as the time constant of the defibrillator system varies, and seeks to minimize the leading edge voltage needed for successful defibrillation. In this model, an optimal phase duration is determined to be:

$$duration = - [ \frac{{τ}_{s} {τ}_{m}}{{τ}_{s} - {τ}_{m}} ] ln ( \frac{{τ}_{m}}{{τ}_{s}} ) \qquad ( 1 )$$

where τₛ is the time constant of the defibrillator system and τₘ is a time constant characteristic of a patient's heart. In one actual embodiment of the invention, τₘ is selected to equal 0.0051 seconds. Since τₛ is the time constant of the defibrillator system including the patient, τₛ is mathematically related to the patient's impedance. As a result, Equation (1) relates an optimal phase duration to patient impedance.

In the preprocessing stage, phase duration values are calculated for possible intermediate elapsed time values t₁ (see FIG. 3), or ranges of elapsed time values T (see FIG. 5) using Equation (1). Since a portion of the defibrillation pulse has already been delivered by the time the intermediate parameter threshold is reached, only the remaining portions of the optimal phase durations need to be stored in the look-up table 82. Given an initial pulse amplitude Vᵢ, an intermediate threshold Vₜ, and an elapsed time t₁ for the defibrillation pulse to reach the intermediate threshold, the time constant τₛ can be calculated. By calculating the time constant τₛ for various patient scenarios, the optimal pulse duration for each scenario, and hence the optimal remaining duration for delivery of the defibrillation pulse after intermediate time t₁ (i.e., from t₁ to t₂ in FIG. 3), can be determined. Of course, determination of the remaining pulse duration using the above factors and Equation (1) can also be performed on-the-fly during the course of defibrillation. In that regard, the defibrillator 10 calculates the time constant τₛ for the present pulse delivery, and using the relationship in Equation (1), calculates the pulse duration, from which the time to the intermediate threshold is subtracted, yielding the remaining time for extending the duration of the pulse (or for the pulse phase, if delivering a multiphasic pulse).

In addition to using the measurement of time that it takes for a defibrillation pulse parameter to reach an intermediate parameter threshold for determining the remaining pulse duration, the time measurement is also useful for calculating the patient's transthoracic impedance. As noted above, the time constant τₛ can be calculated from the initial voltage Vᵢ, the intermediate voltage Vₜ, and the elapsed time t₁. Given the known resistance and capacitance internal to the defibrillator and the time constant τₛ, the patient's impedance may be determined.

A calculation of the patient's impedance is useful for a number of purposes. For example, a high patient impedance calculation may indicate a poor connection of the electrodes 12 and 14 to the patient 16. If the electrode-to-patient connection is good, a high patient impedance calculation may indicate that the patient will require more energy in subsequent pulses, if any, for successful defibrillation. A low patient impedance calculation may indicate the presence of a shunt path between the electrodes that is preventing the energy in the defibrillation pulse from getting to the patient's heart. Accordingly, it is preferable that the defibrillator 10 report the calculation of the patient's impedance so that an operator of the defibrillator (whether a person local to the defibrillator or medical personnel providing instructions or reviewing results from a remote location) can evaluate the quality of the connection of the electrodes to the patient and/or determine appropriate energy settings for the defibrillation therapy.

If the patient's impedance is outside an expected range, e.g., 20-300 ohms, the defibrillator 10 may conclude that an error occurred in the discharge. Equivalently, the defibrillator 10 may conclude that an error occurred if the elapsed time of the discharge is outside an expected time range, as the time it takes to reach an intermediate parameter threshold is directly affected by the patient's impedance. One embodiment of the invention that uses an H-bridge output circuit for delivering the defibrillation pulse, performs a self test, including a test of the H-bridge output circuit, to test its integrity, as described in U.S. Pat. No. 5,873,893, titled “Method and Apparatus for Verifying the Integrity of an Output Circuit Before and During Application of a Defibrillation Pulse,” assigned to the assignee of the present invention and specifically incorporated by reference herein. If a potential discharge error is detected and the self test fails, the defibrillator reports to the operator that it needs to be repaired. If the defibrillator 10 passes the self test, the defibrillator may conclude from the impedance calculation that the operator performed an open or shorted discharge. In that regard, the defibrillator reports an abnormal energy delivery to the operator. In other embodiments of the invention, patient-related parameters other than impedance may be compared to an expected range to determine if the self test failed or if a discharge error has been detected.

While a calculation of patient's impedance is useful to an operator of the defibrillator for determining the energy setting for subsequent pulses or indicating errors in the discharge, the impedance calculation is not used in the invention to adjust any of the discharge parameters, such as discharge duration, voltage, or current. In certain embodiments of the invention, a calculation of the patient's impedance may be used to set the intermediate parameter threshold employed during discharge. Nevertheless, the intermediate parameter threshold is not a discharge parameter. Instead, it is the measurement of elapsed time for an electrical parameter to reach the intermediate threshold that is used to set the remaining duration of the discharge.

The delivery of the defibrillation pulse continues until the remaining time D, determined at block 66, has elapsed. Turning now to FIG. 4B, the defibrillator 10 checks the clock to see if the time to reach the intermediate parameter (from t₀ to t₁ in FIG. 3) plus the remaining time D (from t₁ to t₂ in FIG. 3) has completed, as indicated at decision block 68. Equivalently, if the clock is reset when the intermediate parameter is reached, the defibrillator 10 may check the clock to see if the remaining time D has passed. In either case, when the time extension is completed, the defibrillator 10 truncates the delivery of the first phase, as indicated at block 70.

As noted earlier, if delivering a multiphasic pulse, the present invention may be used for determining the duration of any or all of the phases of the pulse discharge. In terms of the biphasic pulse 40 shown in FIG. 3, the invention is applied only to the first phase of the pulse. Accordingly. at block 72 in FIG. 4B, the delivery time for the entire second phase is determined. The duration of the second phase may be set as a percentage of the duration of the first phase (e.g., 66% or two-thirds of the first phase). Other percentages or procedures for determining the delivery time of the second phase may suitably be used, including measuring elapsed time to an intermediate parameter threshold during the second phase to determine the duration of the second phase.

At block 74, the defibrillator 10 commences delivery of the second phase of the defibrillation pulse (as shown beginning at time t₃ in FIG. 3). The second phase of the biphasic defibrillation pulse shown in FIG. 3 has a polarity opposite that of the first phase. At block 74, the defibrillator 10 observes the clock to measure the duration of the second phase. The time measurement for the second phase may be referenced from the beginning of the second phase (time t₃) or may be referenced from a time point earlier in the pulse (e.g., the beginning of the pulse (time t₀)) with the time t₃ noted for purposes of tracking the delivery time of the second phase. The clock used for measuring the second phase duration may be the same clock used to measure the duration of the first phase, though not required.

At decision block 76, the defibrillator determines if the delivery time of the second phase has completed. If not, the defibrillator continues to discharge energy while measuring the delivery time of the second phase. The defibrillator truncates the delivery of the second phase once its duration is completed, as indicated at block 78. The truncation of the second phase corresponds to time t₄ shown in FIG. 3. Energy remaining in the defibrillation capacitor at the conclusion of the second phase may be dissipated in an energy dump (e.g., discharged into a resistive element) located in or connected to the defibrillator, thus concluding the pulse delivery at end block 80.

While FIG. 4B suggests determining the duration of the second phase after completing the delivery of the first phase, that sequence of actions is not necessary. The second phase duration may be calculated at an earlier time, if desired (e.g., at the same time that the remaining pulse duration D is determined for the first phase in block 66 of FIG. 4A). The sequence of other actions illustrated in FIG. 4A may also be rearranged. For instance, determining the intermediate voltage threshold (block 58) may occur prior to or concurrent with charging the capacitor to the initial charge voltage (block 56).

As noted earlier, in another embodiment of the invention, the intermediate parameter threshold may vary during the defibrillation pulse. For instance, the intermediate parameter threshold may vary as a function of the elapsing time that it takes for the selected electrical parameter to reach the intermediate parameter threshold. Assume, for example, the electrical parameter at issue is capacitor voltage. The intermediate voltage threshold may initially be set at a low level. As the delivery time of the pulse phase increases without the pulse voltage (i.e., capacitor voltage) reaching the intermediate threshold, the intermediate voltage threshold also increases until pulse voltage and the intermediate threshold meet. The increase in the intermediate voltage threshold is not necessarily linear in relationship to the elapsing pulse delivery time. Preferably, the intermediate voltage threshold is adjusted so that the decaying capacitor voltage will meet the threshold at least one millisecond before a desired end of the pulse (or end of the pulse phase in a multiphasic pulse). Using a variable intermediate threshold, a low impedance patient would be subject to a lower intermediate voltage threshold, while a high impedance patient would be subject to a higher intermediate threshold. The intermediate parameter threshold may alternatively be varied based on a measurement of the electrical parameter while it is progressing toward the intermediate parameter threshold. The value of the electrical parameter may be observed at one or more instances during the discharge to adjust the intermediate parameter threshold.

Another alternative embodiment of the invention uses multiple fixed intermediate parameter thresholds. The intermediate threshold used depends on the elapsed time of the energy discharge. This alternative embodiment still involves measuring the time it takes for an electrical parameter related to the energy discharge to reach an intermediate parameter threshold, but the control logic implemented by the defibrillator 10 chooses between two or more intermediate thresholds rather than using a single fixed threshold.

For example, FIG. 6 illustrates a method 84 in which the defibrillator measures the time it takes for the capacitor voltage to reach the first (e.g., the highest) of a set of multiple intermediate voltage thresholds. Beginning at start block 85, the defibrillator determines the initial capacitor voltage (block 86) and charges the capacitor to the initial voltage (block 87), in a manner similar to that described with respect to blocks 54 and 56 in FIG. 4A. At block 88, the defibrillator determines the initial intermediate voltage threshold. The initial intermediate threshold may be selected based on the initial capacitor voltage, the patient's impedance, the defibrillator's energy setting, or other factors relating the pulse delivery. The defibrillator commences delivery of the first phase of the defibrillation pulse at block 89.

The defibrillator continues to deliver the defibrillation pulse to the patient, as indicated at decision block 90, while waiting for the capacitor voltage to reach the initial intermediate voltage threshold, at which time the defibrillator determines the elapsed time that it took to reach the initial threshold, as indicated at block 91. If the elapsed time is within an expected range of time, as indicated at decision block 92, the defibrillator uses the elapsed time to determine the remaining pulse duration at block 93 in a manner similar to that described above with respect to the block 66 (FIG. 4A). If the elapsed time for the capacitor voltage to meet the initial intermediate threshold is not within the expected time range (e.g., the elapsed time is too short), the defibrillator determines the next intermediate voltage threshold at block 94. The next intermediate voltage threshold may be selected from an established set of intermediate voltage thresholds, or may be dynamically determined based, for example, on the initial intermediate voltage threshold. The defibrillator continues the energy discharge while waiting for the capacitor voltage to reach the next intermediate voltage threshold, as indicated at decision block 95. At decision block 96, the defibrillator determines whether the next intermediate voltage threshold determined at block 94 is the last intermediate threshold. If it is not the last intermediate threshold, the defibrillator returns to block 91 to determine the elapsed time that it took to reach the next intermediate threshold. At decision block 92, the defibrillator determines whether the elapsed time is within an expected time range for the next intermediate voltage threshold, and the process is repeated, as needed, for a desired number of iterations.

If the next intermediate threshold was the last intermediate threshold, the defibrillator proceeds to block 97 where it determines the elapsed time that it took to reach the last intermediate threshold. Then, at block 93, the defibrillator determines the remaining time for the pulse delivery. If the elapsed time that it took to reach the last intermediate threshold is within the expected time range, the remaining time may be determined similar to the manner described with respect to block 66 in FIG. 4A. If the elapsed time is outside the time range (e.g., the elapsed time is too short), the defibrillator may extend the delivery time for a default duration and then truncate the pulse. In the last case, the defibrillator preferably produces an advisory alarm to an operator of the defibrillator indicating that the elapsed time of the pulse delivery was outside the expected range. Regardless, the first phase is extended for the determined period of time and the second phase is delivered as shown in FIG. 4B.

While the embodiment of the invention described in FIG. 6 suggests waiting for the electrical parameter to reach an intermediate parameter threshold before evaluating whether the elapsed time is within an expected time range, if the elapsed discharge time exceeds the expected range of time, the defibrillator may proceed automatically to establishing the next intermediate voltage threshold and measuring the elapsed time for the electrical parameter to reach the next intermediate threshold without waiting to reach the first, or initial, intermediate parameter threshold. This process of continuing automatically to the next parameter threshold if the elapsed time exceeds the expected range of time may continue for all subsequent intermediate parameter thresholds until the defibrillator is using the last intermediate threshold. It is also within the scope of the invention for the defibrillator to vary the initial or next intermediate parameter thresholds during the course of pulse delivery. The initial and/or next intermediate parameter thresholds may be varied based on the elapsing time of the pulse delivery.

A block diagram illustrating major components of a defibrillator 10 suitable for carrying out the invention is provided in FIG. 7. The embodiment of the defibrillator 10 shown in FIG. 7 includes a processing unit 100 that is connected to an energy storage device (capacitor) 102 via a charging circuit 104. In alternative embodiments of the invention, the energy storage device may be a battery or other source of energy, located within or external to the energy delivery apparatus of the defibrillator, as desired, consistent with improvements in technology that make such energy storage devices capable of delivering high voltage, high current pulses. The processing unit 100 operates in accordance with programmed instructions stored in a memory 106 that implement the present invention. The processing unit 100 is connected to an output device 107 (e.g., display, data port, printer, speaker, etc.) to provide information and instructions to an operator of the defibrillator.

During the operation of the defibrillator 10, the processing unit 100 controls the charging circuit 104 by a signal on a control line 108 to charge the energy storage capacitor 102 to a desired initial charge level (see block 56 in FIG. 4A). To monitor the charging process, the processing unit 100 is connected to a scaling circuit 110 by a pair of measurement lines 112 and 114, and by a control line 116. The scaling circuit 110 is connected to the positive and negative leads of energy storage capacitor 102 by lines 118 and 120 respectively. A clock 122 is also connected to the processing unit 100. The clock 122 is used for measuring the time that it takes an electrical parameter related to the defibrillation pulse discharge to reach the intermediate parameter threshold, as well as measure the time for delivering the remaining portion of the pulse phase.

The scaling circuit 110 steps down the voltage across the energy storage capacitor 102 to a range that may be accommodated by the processing unit 100. A suitable scaling circuit 110 is briefly described below and in more detail in U.S. Pat. No. 5,873,893, referenced earlier and incorporated herein. The energy storage capacitor 102 can be charged to an initial charge level that depends on the patient's impedance and/or other parameters, such as the energy setting of the defibrillator, as described with respect to blocks 54 and 56 in FIG. 4A.

The size of the energy storage capacitor 102 may suitably fall within a range from 100 uF to 250 uF, though other size capacitors can be used as needed. In order to generate a defibrillation pulse for external application to a patient, the energy storage capacitor is charged to an appropriate voltage, e.g., between 100 volts and 2,200 volts. Small percentage changes in the voltage level of the energy storage capacitor 102 are detected by the scaling circuit 110, which is adjustable to measure different voltage ranges.

After the capacitor 102 is charged to a desired initial voltage, the energy stored in the energy storage capacitor 102 is discharged to the patient 16 in a defibrillation pulse. An output circuit 124 allows the controlled transfer of energy from the energy storage capacitor to the patient in accordance with the present invention. The output circuit 124 includes four switches 126, 128, 130, and 132, each switch on a leg of the output circuit arrayed in the form of an “H” (hereinafter the “H-bridge” output circuit). The four switches 126, 128, 130, 132 can be switched from an off (non-conducting) condition to an on (conducting) condition, and vice versa. Switches 126 and 130 are coupled through a protective component 134 to the positive lead of the energy storage capacitor 102 by a bridge line 136. The protective component 134 limits the current and voltage changes from the energy storage capacitor 102, and has both inductive and resistive properties. Switches 128 and 132 are coupled to the energy storage capacitor 102 by a bridge line 120. An apex line 138 connects the patient 16 to the left side of the H-bridge and a sternum line 140 connects the patient to the right side of the H-bridge. The apex line 138 and the sternum line 140 are connected to electrodes 12 and 14 (depicted in FIG. 1) by a patient isolation relay 142.

The processing unit 100 is connected to the switches 126, 128, 130, and 132 by control lines 144a, 144b, 144c, and 144d, respectively, and to the patient isolation relay 142 by control line 146. Application of appropriate control signals by the processing unit 100 over the control lines causes the switches to be opened and closed, and the output circuit 124 to conduct energy from the energy storage capacitor 102 to the patient.

In one implementation of the present invention, the processing unit 100 is comprised of a 68HC05 microprocessor manufactured by Motorola, which includes on-board analog-to-digital (A/D) converters. The A/D converters are used to convert the analog voltage signals from the scaling circuit 110 to digital data. The scaling circuit 110 includes resistors and amplifiers that scale down and buffer the relatively high capacitor voltage before being provided to the microprocessor. The processing unit 100 activates the on-board A/D converters to sample and digitize the scaled capacitor voltage during discharge of the defibrillation pulse. The microprocessor also uses signals from clock 122 to measure the time from the start of discharge to the time the scaled capacitor voltage reaches the scaled capacitor voltage value corresponding to the intermediate capacitor voltage threshold (e.g., Vₜ in FIG. 3). As noted herein, this time measurement is used to determine the time period for extending the duration of the pulse phase (e.g., by using the time measurement to the intermediate voltage threshold as an index to a look-up table 82 shown in FIG. 5). The processing unit 100 generates control signals for the H-bridge at the appropriate times so that the pulse phase continues for the remaining duration retrieved from the look-up table 82.

In regard to the biphasic pulse 40 shown in FIG. 3, after the first phase is truncated, the processing unit 100 controls the H-bridge to commence delivery of the second phase of the biphasic waveform with a duration that is calculated by the processing unit (e.g., two-thirds of the duration of the first phase). The second phase is also time-terminated, in this case by performing an energy dump at the determined time, thereby fully discharging the capacitor. If desired, the defibrillator 10 may observe the capacitor voltage at other times after initiation of the discharge, e.g., at the end of each phase and/or after the energy dump, to ensure proper operation of the defibrillator.

A suitable construction of the output circuit 124 as discussed above and shown in FIG. 6 is described in greater detail in U.S. Pat. No. 6,041,254, titled “H-bridge Circuit for Generating a High-Energy Biphasic Waveform in an External Defibrillator and Further Including a Protective Component That Has Both Inductive and Resistive Properties,” assigned to the assignee of the present invention and incorporated by reference herein. When the energy storage capacitor 102 is charged to a determined initial charge level and the patient isolation relay 142 is closed, the switches 126 and 128 are switched on so as to connect the energy storage capacitor 102 with the apex line 138 and sternum line 140 for the application of a first phase of the defibrillation pulse to the patient 16. The first phase is preferably a positive pulse that travels from the apex to the sternum of the patient.

After the electrical parameter related to the defibrillation discharge reaches the intermediate electrical parameter threshold (e.g., voltage threshold Vₜ in FIG. 3), and the energy storage device 102 further discharges energy for the determined remaining duration of the first phase, the switch 128 is biased off to truncate delivery of the first phase. Once the switch 128 is biased off, switch 126 also becomes non-conducting. In this implementation of the invention, there is a brief period of time between the end of the first phase and the beginning of the second phase (i.e., between time t₂ and t₃ in FIG. 3), though this is not required. In one actual embodiment of the invention, the time between the phases is approximately 800 microseconds.

After the end of the first phase of the biphasic defibrillation pulse, switches 130 and 132 are switched on to start the second phase of the biphasic pulse. Switches 130 and 132 provide a path to apply a negative polarity defibrillation pulse to the patient 16. At the end of the second phase (i.e., when the determined second phase delivery time has elapsed), the second phase is truncated by switching on switch 126 to provide a shorted path for the remainder of the capacitor energy to drain through switches 126 and 132. After truncation of the second phase, all four of the switches 126 to 132 are switched off and the patient isolation relay 142 is opened. The energy storage capacitor 102 may then be recharged to prepare the defibrillator to apply another defibrillation pulse to the patient 16.

While the above embodiment of the defibrillator 10 contemplates using computer software instructions loaded in memory 106 to direct the processing unit 100 to control the energy discharge (for generating either or both a positive pulse or a negative pulse), an alternative embodiment of the defibrillator 10 may use computer hardware to implement the same tasks. For example, “processing unit” circuitry suitable for use in the invention may include a hardware multiplexer that, switched one way, generates a positive phase pulse, and switched another way, generates a negative phase pulse. The processing unit circuitry may further include comparator circuitry used to compare the actual capacitor voltage with the intermediate capacitor voltage threshold and when the actual capacitor voltage is equal to or less than the intermediate voltage threshold, produce an output signal indicating that the intermediate voltage threshold has been reached. Once the positive pulse is completed (with the appropriate extension of duration, as provided by the present invention), the multiplexer may cause the output circuit to produce a negative pulse. When a pulse width timer indicates that the negative pulse is completed, the timer control signal directs the output circuit to truncate the delivery of the negative pulse. The processing unit circuitry may be located in a single component or distributed across two or more components.

While certain embodiments of the invention described herein are shown using a time measurement to an intermediate voltage threshold to determine the period of time for extending the defibrillation pulse duration, time measurements to other electrical parameter thresholds may be used for the same purpose. For instance, the electrical parameter may be discharge current and the intermediate parameter threshold may be a current threshold. In that regard, the defibrillator waits for the amplitude of the current being delivered to the patient to reach the intermediate current threshold. The measurement of elapsed time for the pulse delivery to reach the intermediate current threshold is then used to determine the remaining discharge time for the pulse phase. Similarly, the electrical parameter may be delivered energy and an energy-based threshold is used. Delivered energy, in this regard, may be a measure of an instantaneous energy being delivered or it may be a summation of the energy delivered over time. The defibrillator uses the elapsed time it takes for the delivered energy to reach the intermediate energy threshold to determine the remaining delivery time of the pulse.

Furthermore, referring to FIGS. 3 and 5, a parameter derived from the elapsed time t₁ rather than t₁ itself may be used as an index to the look-up table 82 for determining the remaining pulse duration. For instance, a suitable look-up table may correlate waveform time constant values τₛ to the remaining duration values D, with the waveform time constant τₛ acting as the index. As discussed earlier, the waveform time constant τₛ can be calculated from the rate of change in the defibrillation pulse amplitude during discharge, which is known from the initial and intermediate voltage levels Vᵢ and Vₜ, and the time t₁ that elapses between them. The look-up table 82 may also correlate other parameters dependent on the time it takes for an electrical parameter to reach the intermediate parameter threshold to determine the remaining duration values D, and use these other parameters as an index to the look-up table. Furthermore, when preparing the look-up table 82 during the preprocessing stage, other embodiments of the invention may use relationships different than that expressed in Equation (1) for determining optimal pulse durations without departing from the invention.

As noted earlier in reference to block 54 of FIG. 4A, U.S. Pat. No. 5,999,852 describes measuring a patient's impedance prior to delivery of the defibrillation pulse using a low-amplitude signal applied to the patient. The patient impedance measured prior to defibrillation may be used to set the intermediate parameter threshold. When the intermediate threshold is met, the elapsed time for the electrical parameter to reach the intermediate threshold is used to determined the remaining pulse duration.

It will be appreciated that other changes may be made without departing from the fundamental concepts of the invention. For instance, it is also within the scope of the invention to use a measurement of patient impedance obtained during a previous defibrillation pulse to set the intermediate parameter threshold for a subsequent defibrillation pulse.

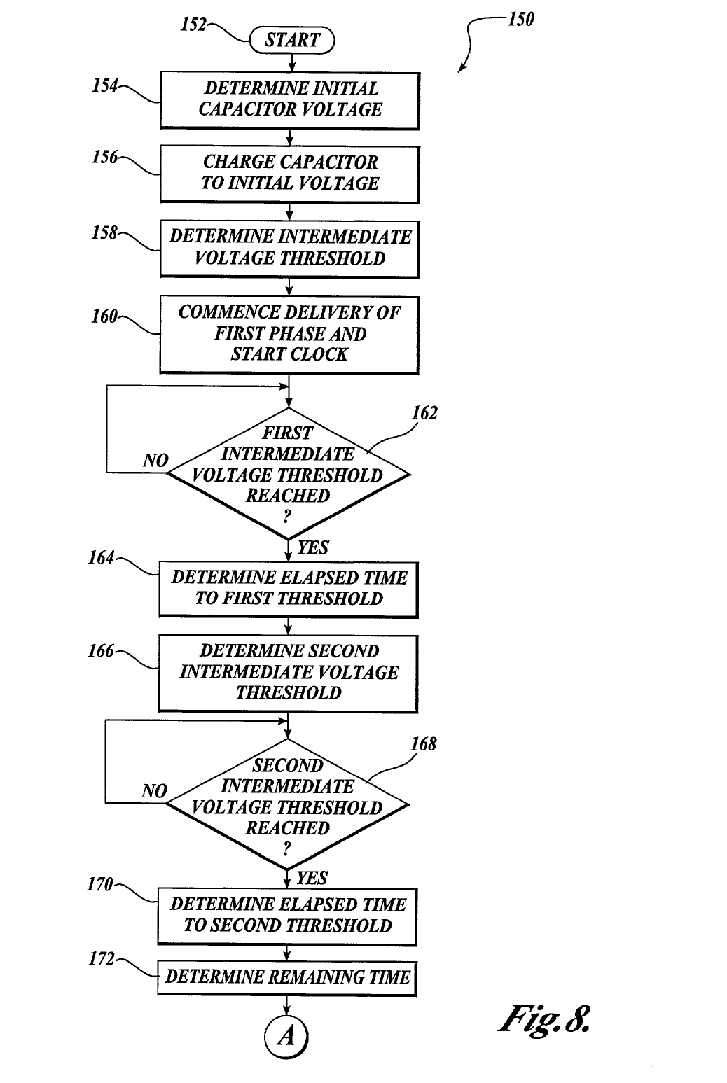

Yet another embodiment of the invention may use a measurement of the time it takes a first electrical parameter related to the discharge to reach a first intermediate threshold to set the value of a second intermediate threshold. The elapsed time for a second electrical parameter (which may or may not be the same as the first electrical parameter) to reach the second intermediate threshold (or other subsequent threshold) is then used to determine the remaining pulse duration. For example, in reference to a method 150 shown at FIG. 8, beginning at a start block 152, the defibrillator determines the initial capacitor voltage (block 154) and charges the capacitor to the initial voltage (block 156), in a manner similar to that described with respect to blocks 54 and 56 in FIG. 4A. At block 158, the defibrillator determines a first intermediate voltage threshold using any one of the techniques discussed herein (e.g., using a fixed percentage of the initial capacitor voltage and/or using other factors, such as the various possible energy settings of the defibrillator, the patient's impedance (measured prior to defibrillation or during an earlier defibrillation pulse), and/or the type of cardiac rhythm detected in the patient). A first intermediate voltage threshold that varies as a function of discharge time may also be used. See, e.g., the discussion provided earlier with respect to block 58 in FIG. 4A.

At block 160, the defibrillator commences delivery of the defibrillation pulse and measures the discharge time of the pulse. The defibrillator continues to deliver the defibrillation pulse to the patient, as indicated at decision block 162, while waiting for the capacitor voltage to decay to the first intermediate voltage threshold, at which time the defibrillator determines the elapsed time to reach the first threshold, as indicated at block 164.

At block 166, the defibrillator determines a second intermediate voltage threshold, preferably as a function of the elapsed time that it took to reach the first threshold. Correlations between the elapsed time to reach the first intermediate threshold and selection of the second intermediate threshold are determined during the preprocessing stage and may be stored in a look-up table in memory.

The defibrillator continues delivering the defibrillation pulse (in this particular example, the first phase of the defibrillation pulse) and waits for the electrical parameter (e.g., capacitor voltage) to reach the second intermediate threshold, as indicated at decision block 168. Once the second intermediate voltage threshold is reached, the defibrillator determines the elapsed time to reach the second threshold at block 170. The elapsed time to the second intermediate threshold is then used at block 172 to determine the remaining time for extending the pulse phase duration. In that regard, a look-up table prepared during the preprocessing stage, such as look-up table 82 shown in FIG. 5, may be used. The delivery of the remainder of the first phase and the delivery of the second phase may then proceed as described in FIG. 4B. The elapsed time between the first and second intermediate thresholds may also be used in determining the remaining pulse duration.

In a further embodiment of the invention, the electrical parameter related to the energy discharge (e.g., capacitor voltage) may be averaged over time. The elapsed time of the discharge is measured to when the averaged electrical parameter reaches the intermediate parameter threshold. Using an average of the electrical parameter, in place of an instantaneous measurement of the electrical parameter, may help overcome parameter measurements that are not perfectly accurate. The electrical parameter may be averaged over a specified time frame using a sliding window or may be averaged from the beginning of the pulse.

In yet a further embodiment of the invention, measurements of the electrical parameter may be integrated, or summed, and the elapsed time for the defibrillator to reach the intermediate parameter threshold is measured to when the summed value of the electrical parameter reaches the intermediate parameter threshold. For example, the electrical parameter related to the energy discharge may be the drop in capacitor voltage at various points in time. The defibrillator observes the drop in capacitor voltage at each point in time and calculates the sum of those values. If the capacitor voltage drops quickly (e.g., for a low impedance patient) the sum will accumulate quickly. If the voltage drops slowly (e.g., for a high impedance patient), the sum will accumulate slowly. The time period for extending the pulse duration, in this embodiment of the invention, is determined based on the time required for the summed capacitor voltage drops to reach the intermediate parameter threshold. Other embodiments of the invention may sum other electrical parameters, for example, the capacitor voltage itself.

Additional embodiments of the invention may use the elapsed time for an electrical parameter to reach an intermediate parameter threshold to determine other conditions for truncating the defibrillation pulse discharge. The condition for truncating the pulse discharge may be, for example, a voltage parameter, current parameter, energy parameter, charge parameter, or time parameter. A voltage parameter, once set based on the measured elapsed time, may be compared against a voltage related to the energy discharge (e.g., capacitor voltage), and when the voltage parameter is met, the energy discharge is truncated. In other exemplary embodiments of the invention, the current parameter may be set, based on the measured elapsed time, relative to the electrical current of the energy discharge; the energy parameter may be set, relative to the amount of energy delivered or yet undelivered to the patient; and the charge parameter may be set relative to the amount of charge in the capacitor that is yet to be discharged or has been discharged to the patient. The time parameter may be, for example, an end time that specifies the time at which the pulse will be truncated, in effect causing the duration of the energy discharge to be extended for a period of time. The period of time for extending the energy discharge may be determined based on the measured elapsed time as discussed with respect to earlier embodiments of the invention.

Viewed collectively, the foregoing exemplary embodiments of the invention describe the manner in which the present invention uses a time measurement observed during pulse delivery to provide more effective and efficient defibrillation therapy. While various embodiments of the invention have been illustrated and described, the embodiments presented herein are provided for purposes of demonstrating the fundamental concepts of the invention. The scope of the invention should not be limited to the specific embodiments discussed herein, but should be determined from the following claims and equivalents thereto.

The embodiments of the invention in which an exclusive property or privilege is claimed are defined as follows:

1. A defibrillator, comprising:
(a) an energy storage device;
(b) electrodes in electrical communication with the energy storage device; and
(c) a processing unit configured to cause the energy storage device to discharge energy in a defibrillation pulse through the electrodes, the processing unit further being configured to:
(i) measure an elapsed time for an electrical parameter related to the discharge to reach a predetermined intermediate parameter threshold; and
(ii) extend the duration of the energy discharge for a period of time that is determined based on the measured elapsed time.

2. The defibrillator of claim 1, wherein the electrical parameter related to the discharge is voltage.

3. The defibrillator of claim 2, wherein the intermediate parameter threshold is predetermined based on a percentage of the voltage of the energy storage device.

4. The defibrillator of claim 1, wherein the electrical parameter related to the discharge is current.

5. The defibrillator of claim 1, wherein the electrical parameter related to the discharge is energy.

6. The defibrillator of claim 1, wherein the electrical parameter related to the discharge is charge.

7. The defibrillator of claim 1, wherein the electrical parameter related to the discharge is averaged, and the elapsed time is measured to when the averaged value of the electrical parameter reaches the predetermined intermediate parameter threshold.

8. The defibrillator of claim 1, wherein the electrical parameter related to the discharge is summed, and the elapsed time is measured to when the summed value of the electrical parameter reaches the predetermined intermediate parameter threshold.

9. The defibrillator of claim 1, wherein the intermediate parameter threshold is predetermined based on a charge level of the energy storage device.

10. The defibrillator of claim 1, wherein the defibrillation pulse is delivered to a patient, and wherein the processing unit is further configured to calculate a parameter related to the patient.

11. The defibrillator of claim 10, wherein the processing unit is further configured to test an aspect of the defibrillator if the calculated patient-related parameter is outside an expected range.

12. The defibrillator of claim 10, further comprising an output device, wherein the processing unit is configured to cause the output device to advise an operator of the defibrillator of a possible error if the calculated patient-related parameter is outside an expected range.

13. The defibrillator of claim 10, wherein the patient-related parameter is impedance.

14. The defibrillator of claim 13, wherein the processing unit calculates the patient's impedance prior to delivery of the defibrillation pulse using a low-amplitude signal transmitted through the patient.

15. The defibrillator of claim 13, wherein the processing unit calculates the patient's impedance using information obtained during delivery of a prior defibrillation pulse to the patient.

16. The defibrillator of claim 13, wherein the patient's impedance is calculated based on an initial value of the electrical parameter, the intermediate parameter threshold, and the measured elapsed time for the electrical parameter to reach the intermediate parameter threshold.

17. The defibrillator of claim 16, further comprising an output device, wherein the processing unit is configured to cause the output device to advise an operator of the defibrillator of the calculated patient impedance.

18. The defibrillator of claim 13, wherein the intermediate parameter threshold is predetermined based on the calculated patient impedance.

19. The defibrillator of claim 1, further comprising a memory, wherein the processing unit is configured to determine the period of time for extending the duration of the energy discharge by using the measured elapsed time as an index to a look-up table containing duration values stored in the memory.

20. A method of delivering a defibrillation pulse, comprising:
(a) discharging energy in a defibrillation pulse from an energy storage device;
(b) during the energy discharge, measuring an elapsed time for an electrical parameter related to the discharge to reach an intermediate parameter threshold;
(c) based on the measured elapsed time, determining a period of time for extending the duration of the energy discharge; and
(d) extending the duration of the energy discharge for the determined period of time.

21. The method of claim 20, wherein the electrical parameter related to the discharge is voltage.

22. The method of claim 21, further comprising setting the intermediate parameter threshold based on a percentage of the voltage of the energy storage device.

23. The method of claim 20, wherein the electrical parameter related to the discharge is current.

24. The method of claim 20, wherein the electrical parameter related to the discharge is energy.

25. The method of claim 20, wherein the electrical parameter related to the discharge is charge.

26. The method of claim 20, wherein the electrical parameter is averaged, and the elapsed time is measured to when the averaged value of the electrical parameter reaches the intermediate parameter threshold.

27. The method of claim 20, wherein the electrical parameter is summed, and the elapsed time is measured to when the summed value of the electrical parameter reaches the intermediate parameter threshold.

28. The method of claim 20, further comprising setting the intermediate parameter threshold based on a charge level of the energy storage device.

29. The defibrillator of claim 20, wherein the defibrillation pulse is delivered to a patient, the method further comprising calculating a parameter related to the patient.

30. The method of claim 29, wherein the energy storage device is part of an energy delivery system, the method further comprising testing an aspect of the energy delivery system if the calculated patient-related parameter is outside an expected range.

31. The method of claim 29, further comprising advising of a possible error if the calculated patient-related parameter is outside an expected range.

32. The method of claim 29, wherein the patient-related parameter is impedance.

33. The method of claim 32, wherein the patient's impedance is calculated based on an initial value of the electrical parameter, the intermediate parameter threshold, and the measured elapsed time for the electrical parameter to reach the intermediate parameter threshold.

34. The method of claim 33, further comprising reporting the calculated impedance of the patient.

35. The method of claim 32, further comprising setting the intermediate parameter threshold based on the calculated patient impedance.

36. The method of claim 32, further comprising calculating the patient's impedance prior to delivery of the defibrillation pulse using a low-amplitude signal transmitted through the patient.

37. The method of claim 32, further comprising calculating the patient's impedance from information obtained during delivery of a prior defibrillation pulse to the patient.

38. The method of claim 20, wherein the period of time for extending the duration of the energy discharge is determined from a look-up table containing duration values.

39. The method of claim 20, further comprising varying the intermediate parameter threshold during the energy discharge based on the elapsing time of the discharge.

40. The method of claim 39, wherein the intermediate parameter threshold is varied according to an anticipated time for the electrical parameter to reach the intermediate parameter threshold and a desired energy discharge duration so that the intermediate parameter threshold is reached at least one millisecond before the end of the desired energy discharge duration.

41. The method of claim 20, further comprising determining the intermediate parameter threshold prior to delivery of the defibrillation pulse, and maintaining the intermediate parameter threshold throughout the delivery of the defibrillation pulse.

42. A defibrillator, comprising:
(a) an energy storage device;
(b) electrodes in electrical communication with the energy storage device; and
(c) a processing unit configured to cause the energy storage device to discharge energy in a defibrillation pulse through the electrodes, the processing unit further being configured to:
(i) measure an elapsed time for an electrical parameter related to the discharge to reach an intermediate parameter threshold that varies during the discharge; and
(ii) extend the duration of the energy discharge for a period of time that is determined based on the measured elapsed time.

43. The defibrillator of claim 42, wherein the intermediate parameter threshold varies based on the elapsing time of the discharge.

44. The defibrillator of claim 43, wherein the intermediate parameter threshold varies according to an anticipated time for the electrical parameter to reach the intermediate parameter threshold and a desired energy discharge duration so that the intermediate parameter threshold is reached at least one millisecond before the end of the desired energy discharge duration.

45. The defibrillator of claim 42, wherein the defibrillation pulse is delivered to a patient, and wherein the processing unit is further configured to calculate a parameter related to the patient.

46. The defibrillator of claim 45, wherein the processing unit is further configured to test an aspect of the defibrillator if the calculated patient-related parameter is outside an expected range.

47. The defibrillator of claim 45, further comprising an output device, wherein the processing unit is configured to cause the output device to advise an operator of the defibrillator of a possible error if the calculated patient-related parameter is outside an expected range.

48. The defibrillator of claim 45, wherein the patent-related parameter is impedance.

49. The defibrillator of claim 48, wherein the patient's impedance is calculated based on an initial value of the electrical parameter, the intermediate parameter threshold, and the measured elapsed time for the electrical parameter to reach the intermediate parameter threshold.

50. The defibrillator of claim 49, further comprising an output device, wherein the processing unit is configured to cause the output device to advise an operator of the defibrillator of the calculated patient impedance.

51. The defibrillator of claim 48, wherein the processing unit is configured to set the initial value of the intermediate parameter threshold based on the calculated patient impedance.

52. The defibrillator of claim 48, wherein the patient's impedance is calculated prior to delivery of the defibrillation pulse based on a low-amplitude signal transmitted through the electrodes.

53. The defibrillator of claim 48, wherein the patient's impedance is calculated from information obtained during delivery of a prior defibrillation pulse to the patient.

54. The defibrillator of claim 42, wherein the processing unit is configured to set the initial value of the intermediate parameter threshold based on a charge level of the energy storage device.

55. The defibrillator of claim 42, wherein the electrical parameter related to the discharge is voltage.

56. The defibrillator of claim 42 wherein the electrical parameter related to the discharge is current.

57. The defibrillator of claim 42, wherein the electrical parameter related to the discharge is energy.

58. The defibrillator of claim 42, wherein the electrical parameter related to the discharge is charge.

59. The defibrillator of claim 42, wherein the electrical parameter related to the discharge is averaged, and the elapsed time is measured to when the averaged value of the electrical parameter reaches the variable intermediate parameter threshold.

60. The defibrillator of claim 42, wherein the electrical parameter related to the discharge is summed, and the elapsed time is measured to when the summed value of the electrical parameter reaches the variable intermediate parameter threshold.

61. The defibrillator of claim 42, further comprising a memory, wherein the processing unit is configured to determine the period of time for extending the duration of the energy discharge by using the measured elapsed time as an index to a look-up table containing duration values stored in the memory.

62. A defibrillator, comprising:
(a) an energy storage device;
(b) electrodes in electrical communication with the energy storage device; and
(c) a processing unit configured to cause the energy storage device to discharge energy in a defibrillation pulse through the electrodes, the processing unit further being configured to:
(i) measure a first elapsed time for a first electrical parameter related to the discharge to reach a first intermediate parameter threshold;
(ii) if the first elapsed time is within a first expected range of time, then extend the duration of the energy discharge for a first period of time that is determined based on the first elapsed time, otherwise continue the energy discharge and measure a second elapsed time for a second electrical parameter related to the discharge to reach a second intermediate parameter threshold; and
(iii) if the second elapsed time is within a second expected range of time, then extend the duration of the energy discharge for a second period of time that is determined based on the second elapsed time, otherwise continue the energy discharge and repeat the process of measuring an elapsed time for an electrical parameter related to the discharge to reach an intermediate parameter threshold until the measured elapsed time is within an expected range of time, at which time the duration of the energy discharge is extended for a period of time that is determined based on the measured elapsed time, or until the process of measuring the elapsed time is repeated for a determined number of iterations, at which time the duration of the energy discharge is extended for a default period of time.

63. The defibrillator of claim 62, wherein the first or second electrical parameter related to the discharge is voltage.

64. The defibrillator of claim 62, wherein the first or second electrical parameter related to the discharge is current.

65. The defibrillator of claim 62, wherein the first or second electrical parameter related to the discharge is energy.

66. The defibrillator of claim 62, wherein the first or second electrical parameter related the discharge is charge.

67. The defibrillator of claim 62, wherein the first or second electrical parameter is averaged, and the first or second elapsed time is measured to when the averaged value of the first or second electrical parameter reaches the first or second intermediate parameter threshold, respectively.

68. The defibrillator of claim 62, wherein the first or second electrical parameter is summed, and the first or second elapsed time is measured to when the summed value of the first or second electrical parameter reaches the first or second intermediate parameter threshold, respectively.

69. The defibrillator of claim 62, wherein the first electrical parameter and the second electrical parameter are the same parameter.

70. The defibrillator of claim 62, wherein the processing unit is further configured to automatically proceed to measuring the second elapsed time if the first elapsed time exceeds the first expected range of time, without waiting for the first electrical parameter to reach the first intermediate parameter threshold.

71. The defibrillator of claim 62, wherein the first or second intermediate parameter thresholds vary during the discharge based on the elapsing time of the discharge.

72. The defibrillator of claim 71, wherein the first or second intermediate parameter thresholds vary according to an anticipated time to reach the first or second intermediate threshold, respectively, and a desired energy discharge duration so that the first or second intermediate threshold is respectively reached at least one millisecond before the end of the desired discharge duration.

73. The defibrillator of claim 71, wherein the defibrillation pulse is delivered to a patient, and wherein the processing unit is configured to calculate an impedance of the patient and set the initial value of the first or second intermediate parameter threshold based on the impedance of the patient.

74. The defibrillator of claim 73, wherein the patient's impedance is calculated prior to delivery of the defibrillation pulse based on a low-amplitude signal transmitted through the electrodes.

75. The defibrillator of claim 73, wherein the patient's impedance is calculated from information obtained during delivery of a prior defibrillation pulse to the patient.

76. The defibrillator of claim 62, wherein the processing unit is configured to set the value of the first or second intermediate parameter threshold based on a charge level of the energy storage device.

77. The defibrillator of claim 62, wherein the defibrillation pulse is delivered to a patient, and wherein the processing unit is further configured to calculate an impedance of the patient.

78. The defibrillator of claim 77, wherein the patient's impedance is calculated based on an initial value of the first or second electrical parameter, the first or second intermediate parameter threshold, and the first or second elapsed time, respectively.

79. The defibrillator of claim 78, further comprising an output device, wherein the processing unit is configured to cause the output device to advise an operator of the defibrillator of the calculated patient impedance.

80. The defibrillator of claim 62, wherein the defibrillation pulse is delivered to a patient, and wherein the first or second intermediate parameter threshold is based on an impedance of the patient.

81. The defibrillator of claim 62, further comprising a memory, wherein the processing unit is further configured to use the first or second elapsed time as an index to a look-up table containing duration values stored in the memory to determine the first or second period of time, respectively, for extending the duration of the discharge.

82. A defibrillator, comprising:
(a) an energy storage device;
(b) electrodes in electrical communication with the energy storage device; and
(c) a processing unit configured to cause the energy storage device to discharge energy in a defibrillation pulse through the electrodes, the processing unit further being configured to:
(i) measure an elapsed time for an initial electrical parameter related to the discharge to reach an initial intermediate parameter threshold;
(ii) while continuing the discharge, repeat a process of determining a subsequent electrical parameter and a subsequent intermediate parameter threshold, each subsequent intermediate parameter threshold being determined based on the elapsed time for the previous electrical parameter to reach the previous intermediate parameter threshold, until a final electrical parameter reaches a final intermediate parameter threshold; and
(iii) extend the duration of the energy discharge for a period of time that is determined based on the elapsed time for the final electrical parameter to reach the final intermediate parameter threshold.

83. The defibrillator of claim 82, wherein the initial or subsequent intermediate parameter threshold varies during the discharge based on the elapsing time of the discharge.

84. The defibrillator of claim 83, wherein the initial or subsequent intermediate parameter threshold varies according to an anticipated time for the initial or subsequent electrical parameter to reach the initial or subsequent intermediate threshold, respectively, and a desired energy discharge duration so that the initial or subsequent intermediate threshold is respectively reached at least one millisecond before the end of the desired discharge duration.

85. The defibrillator of claim 83, wherein the defibrillation pulse is delivered to a patient, and wherein the processing unit is configured to calculate an impedance of the patient and set the initial value of the initial or subsequent intermediate parameter threshold based on the impedance of the patient.

86. The defibrillator of claim 85, wherein the patient's impedance is determined prior to delivery of the defibrillation pulse based on a low-amplitude signal transmitted through the electrodes.

87. The defibrillation pulse of claim 85, wherein the patient's impedance is determined from information obtained during delivery of a prior defibrillation pulse to the patient.

88. The defibrillation pulse of claim 82, wherein the processing unit is configured to set the initial or subsequent intermediate parameter threshold based on a charge level of the energy storage device.

89. The defibrillator of claim 82, wherein the initial or subsequent electrical parameter related to the discharge is voltage.

90. The defibrillator of claim 82, wherein the initial or subsequent electrical parameter related to the discharge is current.

91. The defibrillator of claim 82, wherein the initial or subsequent electrical parameter related to the discharge is energy.

92. The defibrillator of claim 82, wherein the initial or subsequent electrical parameter related to the discharge is charge.

93. The defibrillator of claim 82, wherein the initial or subsequent electrical parameter is averaged, and the initial or subsequent elapsed time is measured to when the averaged value of the initial or subsequent electrical parameter reaches the initial or subsequent intermediate parameter threshold, respectively.

94. The defibrillator of claim 82, wherein the initial or subsequent electrical parameter is summed, and the initial or subsequent elapsed time is measured to when the summed value of the initial or subsequent electrical parameter reaches the initial or subsequent intermediate parameter threshold, respectively.

95. The defibrillator of claim 82, wherein the initial and subsequent electrical parameters are the same parameter.

96. The defibrillator of claim 82, wherein the defibrillation pulse is delivered to a patient, and wherein the processing unit is further configured to calculate an impedance of the patient.

97. The defibrillator of claim 96, wherein the patient's impedance is calculated based on an initial value of the initial or subsequent electrical parameter, the initial or subsequent intermediate parameter threshold, and the elapsed time for the initial or subsequent electrical parameter to reach the initial or subsequent intermediate parameter threshold, respectively.

98. The defibrillator of claim 97, further comprising an output device, wherein the processing unit is configured to cause the output device to advise an operator of the defibrillator of the calculated patient impedance.

99. The defibrillator of claim 96, wherein the initial intermediate parameter threshold is set based on the calculated patient impedance.

100. The defibrillation pulse of claim 82, further comprising a memory, wherein the processing unit is configured to determine the period of time for extending the duration of the energy discharge from a look-up table containing duration values stored in the memory. The discharge duration may be extended from the final intermediate parameter threshold by using the elapsed time for the final electrical parameter to reach the final intermediate parameter threshold as an index to the look-up table.

101. A defibrillator, comprising
(a) an energy storage device;
(b) electrodes in electrical communication with the energy storage device; and
(c) a processing unit configured to cause the energy storage device to discharge energy in a defibrillation pulse through the electrodes until a condition for truncating the energy discharge is met, the processing unit further being configured to:
(i) measure an elapsed time for an electrical parameter related to the discharge to reach an intermediate parameter threshold; and
(ii) determine the condition for truncating the energy discharge based on the measured elapsed time.

102. The defibrillator of claim 101, wherein the condition for truncating the energy discharge is a voltage parameter.

103. The defibrillator of claim 101, wherein the condition for truncating the energy discharge is a current parameter.

104. The defibrillator of claim 101, wherein the condition for truncating the energy discharge is a energy parameter.

105. The defibrillator of claim 101, wherein the condition for truncating the energy discharge is a charge parameter.

106. The defibrillator of claim 101, wherein the condition for truncating the energy discharge is a time parameter.

107. The defibrillator of claim 101, wherein the processing unit is further configured to set the intermediate parameter threshold based on an initial value of a parameter related to the discharge.

108. The defibrillator of claim 101, wherein the processing unit is further configured to set the intermediate parameter threshold based on a charge level of the energy storage device.

109. The defibrillator of claim 101, wherein the defibrillation pulse is delivered to a patient, and wherein the processing unit is further configured to calculate a parameter related to the patent.

110. The defibrillator of claim 109, the processing unit is further configured to test an aspect of the defibrillator if the calculated patient-related parameter is outside an expected range.

111. The defibrillator of claim 109, further comprising output device, wherein the processing unit is configured to cause the output device to advise an operator of the defibrillator of a possible error if the calculated patient-related parameter is outside an expected range.

112. The defibrillator of claim 109, wherein the patient-related parameter is impedance.

113. The defibrillator of claim 112, wherein the patient's impedance is calculated based on an initial value of the electrical parameter, the intermediate parameter threshold, and the measured elapsed time for the electrical parameter to reach the intermediate parameter threshold.

114. The defibrillator of claim 113, further comprising an output device, wherein the processing unit is configured to cause the output device to advise an operator of the defibrillator of the calculated patient impedance.

115. The defibrillator of claim 112, wherein the intermediate parameter threshold is set based on the calculated impedance.

116. The defibrillator of claim 112, wherein the patient's impedance is calculated prior to the energy discharge.

117. The defibrillator of claim 101, wherein the condition for truncating the energy discharge is an end time that causes the duration of the energy discharge to be extended for a period of time that is determined based on the measured elapsed time.

118. The defibrillator of claim 117, further comprising a memory, wherein the processing unit is configured to determine the period of time for extending the duration of the energy discharge from a look-up table containing duration values stored in the memory.

119. The defibrillator of claim 101, further comprising a plurality of intermediate parameter thresholds, wherein the elapsed time for the electrical parameter to reach one of the plurality of intermediate parameter thresholds is measured.

120. The defibrillator of claim 101, wherein the intermediate parameter threshold varies during the energy discharge.

121. The defibrillator of claim 120, wherein the intermediate parameter threshold varies based on the elapsing time of the discharge.

122. The defibrillator of claim 120, wherein the intermediate parameter threshold varies based on a value of the electrical parameter.
